(19) United States
(12) Patent Application Publication
(10) Pub. No.: US 2022/0023244 A1
(43) Pub. Date: Jan. 27, 2022

(54) FORMULATION FOR TOPICAL TREATMENT OF ACUTE WOUNDS

(71) Applicant: King Abdulaziz University, Jeddah (SA)

(72) Inventors: Nabil A. Alhakamy, Jeddah (SA); Osama A. Ahmed, Jeddah (SA); Usama A. Fahmy, Jeddah (SA); Shadab Md, Jeddah (SA)

(21) Appl. No.: 16/934,707

(22) Filed: Jul. 21, 2020

(51) Int. Cl. A61K 31/196; A61K 38/17; A61K 9/14; A61K 9/06; A61P 17/02; A61L 15/44

(52) U.S. Cl. CPC A61K 31/196; A61K 38/1767; A61K 9/14; A61K 9/0014; A61P 17/02; A61L 15/44; A61K 9/06

(57) ABSTRACT

Nanoparticulate, ion-paired complexes of melittin (MLN) and one or more non-steroidal anti-inflammatory drugs, and particularly MLN ion-paired with diclofenac, provide for enhanced wound healing in humans and other animals. Compositions having the nanonparticulates distributed therein or thereon are applied topically to the wound, and the ion-paired complexes have been shown to demonstrate superior wound healing and closure.

### FIELD OF THE INVENTION

This invention generally relates to a topical formulation comprising ion-paired complexes of melittin (MLN), and methods for using the to accelerate the process of wound healing to treat acute wounds.

### BACKGROUND

The skin provides a life-protective barrier between the body and the external environment against physical damage, pathogens, fluid loss, and has immune-neuroendocrine functions that contribute to the maintenance of body homeostasis [1]. The integrity of the structures forming the skin and mucosa deteriorates due to various reasons. A wound is any damage or break in the surface of the skin that occurs as a result of a disruption of the damaging of tissue integrity of the body due to an external trauma. Soft tissue defects or open wounds may result from a variety of events including, but not limited to trauma, burns, diabetic ulcers, severe infections, such as necrotizing fasciitis, venous stasis disease, and pressure ulcerations [2,3].

Complex wounds pose a significant challenge for many health care providers. These wounds are often multifaceted, making treatment tremendously difficult. They represent a substantial burden on the health care industry, with annual costs in North America alone estimated at $10 billion annually [4,5]. Two percent of the general population in the United States (U.S.) has slow or non-healing wounds. Chronic wounds allow longer time for the development of infections and can contribute to the formation of bed sores and ulcers. Therefore, wounds, often also result in patient discomfort and pain, caregiver frustration, individual economic losses, and diminished quality of life [6].

Wound healing is a dynamic process involving many factors and cell types including soluble mediators, blood cells, fibroblasts, endothelial cells, and extracellular matrix. Normal wound healing is divided into several sequential phases that overlap in space and time: homeostasis, inflammation, granulation tissue formation, and tissue remodeling. Traditional methods of treating wounds to promote healing include keeping the wound clean and protecting the wound from harmful bacterial contamination. There have also been a number of methods developed that include application to the wound of a wound healing agent to facilitate wound healing. The different agents that are used for wound healing and treatment includes antibiotics and antiseptics, desloughing agents (chemical debridement, e.g., hydrogen peroxide, eusol and collagenase ointment), wound healing promoters, some substances such as tissue extracts, vitamins, and minerals and a number of plant products [7-9].

Local application of therapeutic compounds either to the skin, or into the systemic circulation after passage through the skin, offers many advantages over oral and injectable drug delivery. These potential advantages include avoidance of hepatic first-pass metabolism, improved patient compliance and ease of application to the skin. Topical preparations are used for the localized effects at the site of their application by virtue of drug penetration into the underlying layers of skin or mucous membranes.

U.S. Patent Application 2019/0008907 to Al-Waili describes compositions containing bee venom or melittin for healing wounds. The composition may be administered topically. It is disclosed that bee venom/melittin was known to have anti-inflammatory, antibacterial, anti-rheumatic, and antioxidant activity and has previously been used to treat skin diseases.

U.S. Patent Application 2018/0028713 to Agarwal et al. describes nanoscale microsheets containing bioactive agents for wound healing. The bioactive agent may be an antimicrobial such as melittin or an NSAID. The microsheet can be applied to the skin. The disclosed microsheets have combination of a flexible polymer comprising a low surface energy surface and a nanoscale polymer layer adjacent to and in contact with the low surface energy surface. Hence, there is still a need to provide topical compositions of therapeutic agents that can improve and accelerate wound healing.

### SUMMARY

Bee venom is a protein complex consisting of melittin (MLN), phospholipase A2, apamin, and hyaluronidase. Among these components, MLN, a 26 amino acid peptide with the following sequence: Gly-Ile-Gly-Ala-Val-Leu-Lys-Val-Leu-Thr-Thr-Gly-Leu-Pro-Ala-Leu-Ile-Ser-Trp-Ile-Lys-Arg-Lys-Arg-Gln-Gln-NH2 [SEQ ID NO. 1] is the principal component of bee venom. The molecular weight of MLN is about 3 kD. It has a high aqueous solubility, which has potential for use as a biologically active peptide drug. Bee venom had been found to have good effects on the immune system, cardiovascular system, blood, and anti-tumor effects. Bee Venom therapy has also been practiced in traditional medicine. Among the Bee Venom components, MLN is commonly used in the treatment of arthritic disorders, such as rheumatoid arthritis and osteoarthritis. Previous reports have shown honey bee venom (MLN is main constituent) is effective in burns and scarring [10,11].

Embodiments of the present invention relate to complexing melittin and diclofenac sodium based on attraction of different electrostatic charges on both compounds in a way that produces particles, preferably in the nano size range. In the practice of the invention, MLN along with other therapeutic agents can be used to accelerate the process of acute wound healing and thereby for treatment of the same. In particular, complexes of MLN with other therapeutic agents can be used to accelerate the process of wound healing. More particularly, complexes of MLN with Non-steroidal anti-inflammatory drugs can be used to accelerate the process of wound healing.

Accordingly, embodiments of the invention include complexes of MLN with the non-steroidal anti-inflammatory drugs. More specifically, an exemplary complex includes MLN with diclofenac sodium (DCL).

Additional embodiments of the invention include use of the pharmaceutical compositions for topical application to wounds, where the composition include a plurality of particles having complexes of melittin with non-steroidal anti-inflammatory drugs.

In a particular exemplary embodiment a pharmaceutical composition for topical application to wounds comprises a plurality of particles having complexes of melittin with diclofenac and pharmaceutically acceptable salt thereof.

According to the invention, topical treatment methods which employ the compositions can accelerate the process of wound healing. In one aspect, the present invention discloses use of complexes of MLN with non-steroidal anti-inflammatory agents for the treatment of wounds. In further aspect, the present invention discloses use of complexes of MLN with DCL for the treatment of wounds.

### DESCRIPTION OF THE DRAWINGS

The patent or application file contains at least one drawing executed in color. Copies of this patent or patent application publication with color drawings will be provided by the Office upon request and payment of the necessary fee.

FIG. 1 is a transmitted electron microscopic (TEM) image of an MLN-DCL complex;

FIG. 2 is a set of photographs showing wound closure in rats with topical application of different treatments at days 0, 3, 7 and 10.

FIG. 3 is a graph showing wound contraction % at day 10, Data are expressed as mean±SD and analyzed one-way ANOVA followed by Tukey's test, **** Significantly different at p<0.0001

FIG. 4 is a graph showing the effect of different treatments on mRNA expression of Collagen 1A1. Data are expressed as mean±SD and analyzed by one-way ANOVA followed by Tukey's test. ** Significantly different at p<0.05, *** Significantly different at p<0.001, **** Significantly different at p<0.0001

FIG. 5 is a graph showing the effect of different treatments on mRNA expression of collagen 4A1. Data are expressed as mean±SD and analyzed by one-way ANOVA followed by Tukey's test. ** Significantly different at p<0.05, *** Significantly different at p<0.001, **** Significantly different at p<0.0001

FIG. 6 is a graph showing the effect of different treatments on wounded skin content of hydroxyproline. Data are expressed as mean±SD and analyzed by one-way ANOVA followed by Tukey's test. ** Significantly different at p<0.05, *** Significantly different at p<0.001, **** Significantly different at p<0.0001

### DETAILED DESCRIPTION

As used herein the term “treating” or “treatment” broadly includes any kind of treatment activity, including the diagnosis, cure, mitigation, or prevention of disease in man or other animals, or any activity that otherwise affects the structure or any function of the body of man or other animals.

The term “complex” or “complexes” or “ion-paired complexes” mean the interaction between two molecules or portions of the same molecule through noncovalent interactions such as coordination bonds, electrostatic interactions, hydrogen bonding interactions, and hydrophobic interactions and/or through ionic interactions.

As used herein the term “melittin” or “(MLN)” refers to a 26 amino acid peptide with the following sequence: Gly-Ile-Gly-Ala-Val-Leu-Lys-Val-Leu-Thr-Thr-Gly-Leu-Pro-Ala-Leu-Ile-Ser-Trp-Ile-Lys-Arg-Lys-Arg-Gln-Gln-NH2 (SEQ ID No. 1). However, since, it is principal component of the bee venom, MLN could also mean to represent bee venom.

The present invention is directed towards the novel ion-paired complexes of MLN with non-steroidal anti-inflammatory drugs (NSAIDs). As an example, DCL is used as therapeutic agent to form the complex with MLN. Other NSAIDs may be complexed with MLN within the practice of this invention.

Unless otherwise indicated, any reference to a compound herein, such as the NSAID DCL or any other NSAID, by structure, name, or any other means, includes pharmaceutically acceptable salts, alternate solid forms, such as polymorphs, solvates, hydrates, enantiomers, tautomers, deuterium-modified forms, or any other chemical species, such as precursors, prodrugs, or any other chemical species that may rapidly convert to a compound described herein under conditions in which the compounds are used as described herein.

An exemplary complex according to the present invention comprises MLN and DCL in the range of 0.01:1 to 100:1 by molar ratios. Preferably, the molar ratio of MLN and DCL in the present complex is 0.1:1 to 1:0.1 and even more preferably it is 1:1.

The complexes can be prepared by a variety of techniques For example, complexes containing MLN and DCL may be produced by making a solution of MLN and DCL, where the MLN and DCL are mixed at suitable concentrations. After the complex dispersion is obtained, the solvent is removed by, for example, a lyophilization process to get the complex. Preferably, the complexes are in the form of nanoparticles (e.g., 5-700 nm).

Wounds on humans and animals (e.g., mammals such as dogs and cats) may be effectively treated using the complexes described herein. In a preferred embodiment, the process involves topical application of a pharmaceutical compositions comprising ion-paired complexes of the MLN and DCL (or other NSAID) to the wound surface. It has been found that the application of MLN-DCL complex accelerates the wound healing process.

The complexes of the present invention may be given in the form of topical solutions and are applied to the affected areas. Other excipients which are normally added to such formulations can be included in the present compositions. In one embodiment, the topical formulation of the present invention may take the form of a cream, a lotion, an ointment, a hydrogel, a colloid, a gel, a foam, an oil, a milk, a suspension, a wipe, a sponge, a solution, an emulsion, a paste, a patch, a pladget, a swab, a dressing, a spray or a pad. Preferably, the topical formulation of the present invention are provided as a hydrogel formulation.

The topical formulation may comprise one or more pharmaceutically acceptable carriers, and the carriers may be solid (e.g., a paste or wipe), or a fluid (e.g., gel or ointment). Examples of the pharmaceutically acceptable carriers that are usable in the context of the present invention include carrier materials such as a solvent, a stabilizer, a solubilizer, a filler, a tonicity enhancing agent, a structure-forming agent, a suspending agent, a dispersing agent, a chelating agent, an emulsifying agent, an anti-foaming agent, an ointment base, an emollient, a skin protecting agent, a gel-forming agent, a thickening agent, a pH adjusting agent, a preservative, a penetration enhancer, a complexing agent, a lubricant, a demulcent, a viscosity enhancer, a bioadhesive polymer, or a combination thereof. Examples of solvents are water or purified water, alcohols (e.g., ethanol, benzyl alcohol), vegetable, marine and mineral oils, polyethylene glycols, propylene glycols, glycerol, and liquid polyalkylsiloxanes. Inert diluents or fillers may be included in the composition, such as, sucrose, sorbitol, sugar, mannitol, microcrystalline cellulose, starches, calcium carbonate, sodium chloride, lactose, calcium phosphate, calcium sulfate, or sodium phosphate. Buffering agents may be included in the composition. Examples of buffering agents include citric acid, acetic acid, lactic acid, hydrogenophosphoric acid, diethylamine, sodium hydroxide and tromethane (i.e., tris(hydroxymethyl)aminomethane hydrochloride). Other ingredients which may be included in the compositions include suspension agents. Suitable suspending agents are, for example, naturally occurring gums (e.g., acacia, arabic, xanthan, and tragacanth gum), celluloses (e.g., carboxymethyl-, hydroxyethyl-, hydroxypropyl-, and hydroxypropylmethyl-cellulose), alginates and chitosans.

Examples of dispersing or wetting agents that may be included in the compositions are naturally occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate). Preservatives may be added to a topical composition of the invention to prevent, for example, microbial contamination that can affect the stability of the formulation and/or cause infection in the patient. Suitable examples of preservatives include parabens (such as methyl, ethyl, propyl, /p-hydroxybenzoate, butyl, isobutyl, and isopropylparaben), potassium sorbate, sorbic acid, benzoic acid, methyl benzoate, phenoxyethanol, bronopol, bronidox, MDM hydantoin, iodopropynyl butylcarbamate, benzalconium chloride, cetrimide, and benzylalcohol. Examples of chelating agents that may be included in the compositions include sodium EDTA and citric acid.

Examples of gel bases or viscosity-increasing agents that may be included in the topical compositions of the present invention include liquid paraffin, polyethylene, fatty oils, colloidal silica or aluminum, glycerol, propylene glycol, propylene carbonate, carboxyvinyl polymers, magnesium-aluminum silicates, hydrophilic polymers (such as, for example, starch or cellulose derivatives), water-swellable hydrocolloids, carragenans, hyaluronates, alginates, and acrylates. Ointment bases suitable for use in the compositions of the present invention may be hydrophobic or hydrophilic, and include paraffin, lanolin, liquid polyalkylsiloxanes, cetanol, cetyl palmitate, vegetal oils, sorbitan esters of fatty acids, polyethylene glycols, and condensation products between sorbitan esters of fatty acids, ethylene oxide (e.g., polyoxyethylene sorbitan monooleate), polysorbates, white petrolatum and white wax. Examples of humectants that may be included in the compositions are ethanol, isopropanol glycerin, propylene glycol, sorbitol, lactic acid, and urea. Suitable emollients include cholesterol and glycerol. Examples of skin protectants that may be included in the compositions include vitamin E, allatoin, glycerin, zinc oxide, vitamins, and sunscreen agents.

Thickening agents are generally used to increase viscosity and improve bioadhesive properties of pharmaceutical or cosmetic compositions. These agents may be used in some embodiments of the present invention. Examples of thickening agents include, but are not limited to, celluloses, polyethylene glycol, polyethylene oxide, naturally occurring gums, gelatin, karaya, pectin, alginic acid, povidone, and Carbopol® polymers. Particularly interesting are thickening agents with thixotropic properties (i.e., agents whose viscosity is decreased by shaking or stirring). The presence of such an agent in a composition allows the viscosity of the composition to be reduced at the time of administration to facilitate its application to the skin and, to increase after application so that the composition remains at the site of administration. Bioadhesive polymers maybe used in various embodiments of the invention (e.g., on a bandage, in a gel, etc.) are useful to hydrate the skin and enhance its permeability. Bioadhesive polymers can also function as thickening agents. Examples of bioadhesive polymers include, but are not limited to, pectin, alginic acid, chitosan, polysorbates, poly(ethyleneglycol), oligosaccharides and polysaccharides, cellulose esters and cellulose ethers, and modified cellulose polymers.

Permeation enhancing agents are vehicles containing specific agents that affect the delivery of active components through the skin. Such agents may be employed in various embodiments of the invention. Permeation enhancing agents are generally divided into two classes: solvents and surface active compounds (amphiphilic molecules). Examples of solvent permeation enhancing agents include alcohols (e.g., ethyl alcohol, isopropyl alcohol), dimethyl formamide, dimethyl acetamide, dimethyl sulfoxide, 1-dodecylazocyloheptan-2-one, N-decyl-methylsulfoxide, lactic acid, N,N-diethyl-m-toluamide, N-methyl pyrrolidone, nonane, oleic acid, petrolatum, polyethylene glycol, propylene glycol, salicylic acid, urea, terpenes, and trichloroethanol. Surfactant permeation enhancing agents may be nonionic, amphoteric, cationic, or zwitterionic. Suitable nonioinic surfactants include poly(oxyethylene)-poly(oxypropylene) block copolymers, commercially known as poloxamers; ethoxylated hydrogenated castor oils; polysorbates, such as Tween 20 or Tween 80. Amphoteric surfactants include quaternized imidazole derivatives, cationic surfactants include cetypyridinium chloride, and zwitterionic surfactants include the betaines and sulfobetaines. Other examples of suitable permeation enhancers include pentadecalactone, 2-pyrrolidine, 1-dodecal-azacycloheptane-2-one, calcium thioglycolate, hexanol, derivatives of 1,3-dioxanes (i.e., 1,3-dioxacyclohexanes) and 1,3-dioxalanes (i.e., 1,3-dioxacyclopentanes), 1-N-dodecyl-2-pyrrolidone-5-carboxylic acid, 2-pentyl-2-oxo-pyrrolidineacetic acid, 2-dodecyl-2-oxo-1-pyrrolidineacetic acid, and 1-azacycloheptan-2-one-2-dodecylacetic acid among others.

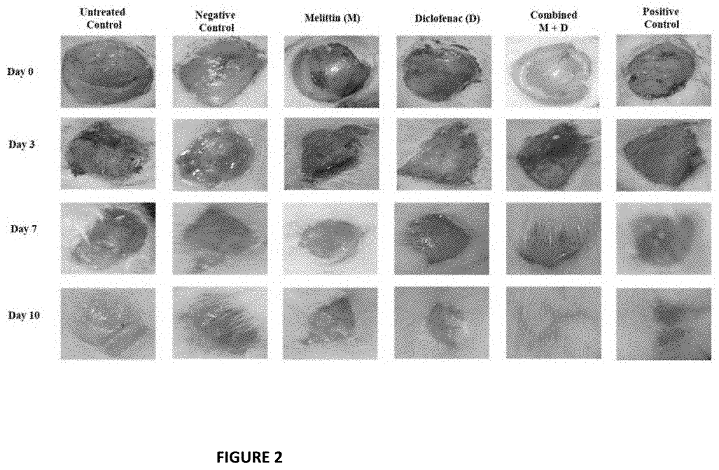

The healing activity of the present formulation was evaluated in the rat animal model. Data, as presented in FIG. 2 indicate that local application of ion pair complex of diclofenac and melittin preparation showed unexpectedly synergistic wound contraction % activity as compared to control treatment. At day 10, rats at the ion-paired complex of diclofenac and melittin treatment group showed almost complete healing. However, wound contraction in animals allocated in only melittin or only diclofenac groups showed values less than 50%. The wound closure experiments usually last for 2-3 weeks, but the ion pair complex of diclofenac and melittin showed unexpectedly complete wound closure at day 10 while melittin or diclofenac sodium failed to do so individually. This data indicates that ion-paired complex of diclofenac and melittin preparation exhibits superior wound healing activity as compared to the positive control treatment.

The following examples further illustrate the invention but should not be construed as in any way limiting its scope. In particular, the processing conditions are merely exemplary and can be readily varied by one of ordinary skill in the art. For example, the complexes of MLN may include one or NSAIDs, and may be the same or different from DCL.

### EXAMPLES

### Example-1 Preparation of MLN-DCL Ion-Pairing Complex

Solutions containing suitable concentrations (0.5 to 10 mM) of MLN were combined with DCL (5 mM) solutions at molar ratios from 0.1:1, 2:1 and 1:0.1 to obtain the desired DCL:MLN ratio. Particle size was measured by the Malvern zetasizer whereas morphology of the MLN-DCL complex was studied using TEM (JEOL-JEM-1011 (JEOL-Tokyo, Japan). The results are summarized in Table 1.

TABLE 1
Particle size and zeta potential values
of the prepared MLN-DCL ion pair complex

| Formula | DCL:MLN (mM) | Size (nm) | Zeta potential (mV) |
|---|---|---|---|
| F1 | 0.1:1 | 193 ± 23.6 | 32.4 ± 1.2 |
| F2 | 1:0.5 | 104 ± 22.6 | 28.3 ± 2.1 |
| F3 | 1:0.1 | 268 ± 37.1 | 0.2 ± 0.01 |

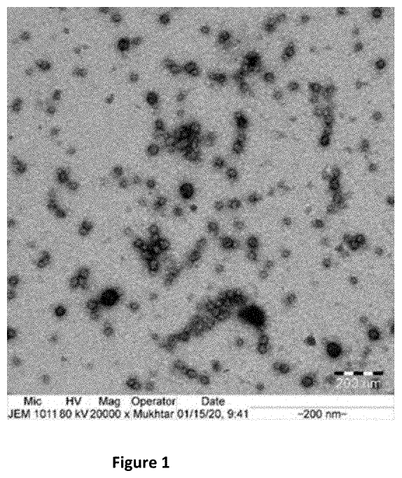

Particle size and shape were further confirmed by the TEM image (see FIG. 1). TEM image revealed that the MLN-DCL ion-pairing complex were spherical particles in shape and in nanometer size comparable to the results obtained by particle size analyzer. Data of DCL:MLN with 1:0.5 mM gave the smallest size; it was reported before that particle sizes around 100 nm achieve maximum cellular uptake. Accordingly, formula F2 was selected for the in vivo investigation of wound healing based on promising particle size and zeta potential results. In addition, ratios outside these ranges showed formation of large aggregates of formed complex.

### Example 2: The Topical Formulations Containing MLN and DCL

A topical formulation containing MLN and DCL was prepared as follows:

Hydrogel formulations were prepared using Hydroxypropyl methyl cellulose (HPMC). HPMC was used as a gelling agent in concentration of 1.5% w/v. HPMC was dissolved in distilled water under magnetic stirring for 30 min at room temperature. MLN-DCL complex was added to the stirred solution at a concentration of (0.6% w/v). The hydrogel was left to swell for at least 24 h at 4° C. before further experiments. DCL alone Hydrogel with was prepared in a concentration of (0.1% w/v) with the same procedure described for the complex hydrogel. In addition, MLN alone Hydrogel with was prepared in a concentration of (0.5% w/v) with the same procedure described for the complex hydrogel. The viscosity of the prepared HPMC hydrogels was determined by using Brookfield viscometer (Brookfield Engineering Laboratories; Stoughton, Mass., USA). The results of the prepared HPMC hydrogels showed a viscosity of 202.5±38.7 cp.

### Example-3: In Vivo Wound Healing Investigation

The Research Ethics Committee, Faculty of Pharmacy, King Abdulaziz University officially approved the experimental protocol (Reference # PH-126-41). Thirty-six male Wistar rats (200-230 g) were used for the study, courtesy of the Animal Facility, King Abdulaziz University, Jeddah, Saudi Arabia. Animals were maintained on a 12-h light-dark cycle and a fixed temperature of 22±2° C.

Wounding by Excision and Animal Treatment

Rats were anesthetized, then dorsal surface shaved, skin cleaned with 70% ethanol. Then, a full-thickness acute excision circle of 1 cm in diameter was engraved on rats' dorsal surface. Then, wounds were disinfected after the excision was completed. Lidocaine hydrochloride (2%) containing 1:80,000 epinephrine (4.4 mg/kg) was injected subcutaneously near the wound area immediately after wounding to reduce pain and bleeding. The wounds were then covered with sterile Vaseline Gauzes dressings and changed once daily.

Wounded rats were randomly divided into six groups (6 each) as follows:

Group 1: Untreated control rats with no treatment.
Group 2: Negative control rats received topical daily Hydroxypropyl-methyl cellulose (HPMC) based hydrogel (1.5% w/v) on the wound area
Group 3: HPMC (1.5% w/v) based hydrogel preparation of melittin (0.5% w/v) on the wound area
Group 4: HPMC (1.5% w/v) based hydrogel of diclofenac sodium (0.1% w/v) on the wound area.
Group 5: HPMC (1.5% w/v) based hydrogel melittin combined with diclofenac in form of nanoparticles (0.6% w/v).
Group 6: Positive control rats treated with 0.5 g of Mebo® ointment (Gulf Pharmaceutical Industries Julphar, Ras Al Khaimah, UAE) on the wound area. The ointment contained f3-Sitosterol, baicalin, and berberine as active ingredients in a base of beeswax and sesame oil.

All treatments were applied topically once daily for 10 consecutive days. Wounds were assessed and photographed at day 0, 3, 7 and 10. At day 10, animals in all groups were sacrificed by ether overdose and the skin in the wound area was dissected out. Part of the skin from each animal was flash frozen in liquid nitrogen and kept in −80° C. for subsequent analyses.

Wound Measurement

The percentage of wound contraction was determined by utilizing the following formula:

$$Wound contraction ( \% ) = ( \frac{\begin{matrix}Day 0 wound diameter - \\ Day 10 wound diameter\end{matrix}}{Day 0 wound diameter} ) \times 100 \qquad equation ( 1 )$$

The average wound healing was indicated by the shedding of scars and complete re-epithelialization of the wound bed.

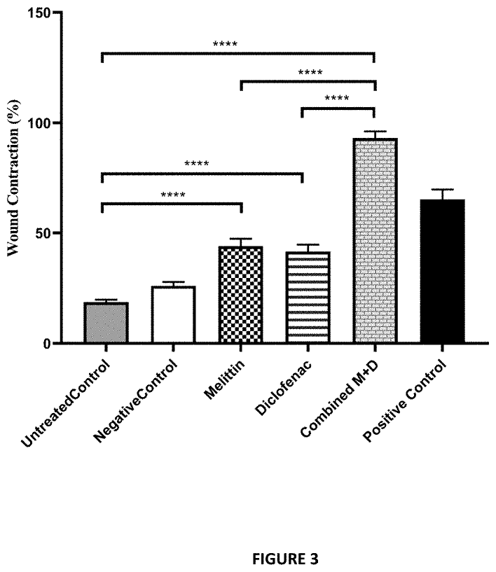

Data in FIGS. 2 and 3 indicate that local application of combined M+D preparation (i.e., particles of MLN complexed with DCL in an ointment) showed unexpectedly synergistic wound contraction % activity as compared to either treatment alone. At day 10, rats at the combined M+D treatment group showed almost complete healing. However, wound contraction in animals allocated in the M or D groups showed values less than 50%. The wound closure experiments usually last for 2-3 weeks, but the complexes of M+D sodium showed unexpectedly complete wound closure at day 10 while melittin or diclofenac sodium failed to do so individually, as indicated in FIGS. 2 and 3. It is noteworthy to report that combined M+D preparation exhibited superior wound healing activity as compared to the positive control treatment (FIG. 3).

### Example-4: Preparation of Tissue Homogenate and Determination of Hydroxyproline

Skin tissues from wound area were carefully rinsed with ice cooled saline and gently blotted between filter papers. Ten percent of homogenates were prepared in ice cooled phosphate-buffered saline (50 mM potassium phosphate, pH 7.4) at 4° C. Then, the homogenates were centrifuged at 10,000 g for 20 min at 4° C. and supernatants were collected. Commercially available kits were used to determine hydroxyproline (catalog #ab222941) and total proteins (ab219272) (ABCAM, Cambridge, UK).

### RT-qPCR in Excised (Healing/Healed) Tissue

Skin tissues from wound area were homogenized in an ultrasonic probe. RNA was extracted utilizing a nucleic acid extraction kit (NucleoSpin®) purchased from Macherey-Nagel GmbH & Co. KG, Duerin, Germany. The purity (A260/A280 ratio) and the concentration of RNA were obtained using spectrophotometry (Dual-Wavelength Beckman, Spectrophotometer, USA). Reverse transcription was undertaken to construct a cDNA library using a High-Capacity cDNA Reverse Transcription Kit (Applied Biosystems, Foster City, Calif., USA). PCR amplification reactions were then performed using a Taq PCR Master Mix Kit (Qiagen, Valencia, Calif., USA) using the primers indicated in Table 2. After the RT-PCR run, the data were expressed in the cycle threshold (Ct). The PCR datasheet included the Ct values of the assessed genes (Coolagen 1A1 & collagen 4A1) and the housekeeping gene (GAPDH). The relative quantitation (RQ) of each target gene was quantified according to the calculation of delta-delta Ct (AACt).

TABLE 2
Primers sequences:

| | Forward | Reverse | Gene bank |
|---|---|---|---|
| Co11A1 | ATCAGCCCA | CGCAGGAAGG | NM_ |
| | AACCCCAAG | TCAGCTGGA | 053304.1 |
| | GAGA | TAG | |
| | SEQ ID NO. 2 | SEQ ID NO. 3 | |
| Col4A1 | CGCTGCGAAG | AAAAGGGTGAT | NM_ |
| | GGTGATTGT | GCTGGAGAAC | 001135009.1 |
| | SEQ ID NO. 4 | SEQ ID NO. 5 | |
| GAPDH | CCATTCTTCC | TGTTGCTGTAG | NM_ |
| | ACCTTTGAT | CCATATTCA | 017008.4 |
| | GCT | TTGT | |
| | SEQ ID NO. 6 | SEQ ID NO. 7 | |

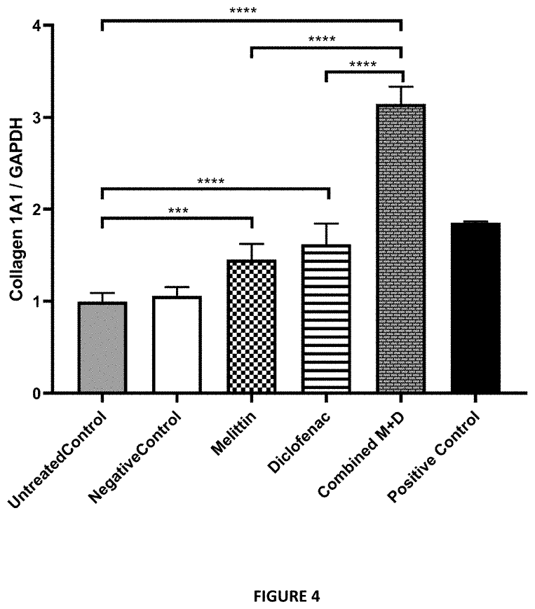

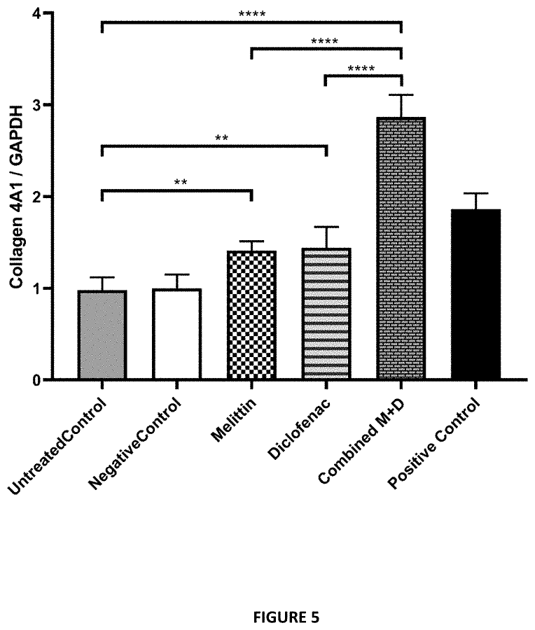

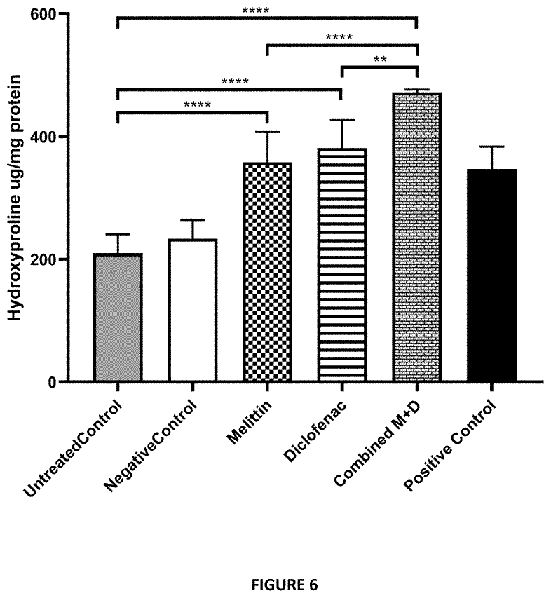

Data in FIGS. 4-6 indicate that the combined M+D treatment significantly enhanced wound healing via up-regulation of Collagen 1A1 and Collagen 4A1 and collagen formation as compared to other treatment groups. Also, M+D treatment significantly enhanced wound healing via hydroxyproline content (marker of collagen formation) as compared to other treatment groups.

### Acknowledgement:

This project was funded by Science and Technology Unit-King Abdulaziz University-Kingdom of Saudi Arabia-award number (UE-41-108).

### REFERENCES

1. Cañedo-Dorantes, L.; Cañedo-Ayala, M. Skin Acute Wound Healing: A Comprehensive Review. hindawi.com 2019.
2. White, R.; nursing, K. C.-B. journal of; 2003, undefined Interventions to avoid maceration of the skin and wound bed. magonlinelibrary.com 2003, 12, 1186-1201.
3. James, W.; Elston, D.; Berger, T. Andrew's Diseases of the Skin E-Book: Clinical Dermatology; 2011;
4. Mulder, M. Basic principles of wound care; 2002;
5. Gottrup, F.; Apelqvist, J.; Bjansholt, T.; Cooper, R.; Moore, Z.; Peters, E. J. G.; Probst, S. EWMA document: Antimicrobials and non-healing wounds evidence, controversies and suggestions. J. Wound Care 2013, 22, S1-S89.
6. Gonzalez, A.; Costa, T.; . . . Z. A.-A. brasileiros; 2016, undefined Wound healing-A literature review. SciELO Bras.
7. Eming, S.; Brachvogel, B.; Odorisio, T.; histochemistry, M. K.-P. in; 2007, undefined Regulation of angiogenesis: wound healing as a model. Elsevier.
8. Greaves, N.; Ashcroft, K.; . . . M. B.-J. of dermatological; 2013, undefined Current understanding of molecular and cellular mechanisms in fibroplasia and angiogenesis during acute wound healing. Elsevier.
9. Reinke, J.; research, H. S.-E. surgical; 2012, undefined Wound repair and regeneration. karger.com.
10. Hozzein, W. N.; Badr, G.; Badr, B. M.; Allam, A.; Ghamdi, A. Al; Al-Wadaan, M. A.; Al-Waili, N. S. Bee venom improves diabetic wound healing by protecting functional macrophages from apoptosis and enhancing Nrf2, Ang-1 and Tie-2 signaling. Mol. Immunol. 2018, 103, 322-335.
11. Han, S. M.; Lee, K. G.; Yeo, J. H.; Kweon, H. Y.; Woo, S. O.; Lee, M. Y.; Baek, H.; Park, K. K. Inhibitory effect of bee venom against ultraviolet B induced MMP-11 and MMP-3 in human dermal fibroblasts. J. Apic. Res. 2007, 46, 94-98.

1-7. (canceled)

8. A method of accelerating closure and healing of an acute wound, comprising
topically applying to a surface of the acute wound at least once daily a therapeutically effective amount of a pharmaceutical composition comprising a carrier, and a plurality of nanoparticles having complexes of melittin with one or more non-steroidal anti-inflammatory drugs (NSAIDS) distributed therein or thereon, wherein said complexes are ion-pair complexes and wherein a molar ratio of the melittin to NSAID in the nanoparticles ranges from 0.1:1.0 to 1.0:0.1; and
wherein therapeutic efficacy of closure and healing of the acute wound is determined by measuring wound closure and/or wound contraction percentage.

9. The method of claim 8 wherein the one or more NSAIDS comprise diclofenac and pharmaceutically acceptable salts, solvates, or hydrates thereof.

10. The method of claim 9 wherein the molar ratio of the melittin to diclofenac is 0.5:1.0.

11. The method of claim 8 wherein the carrier is a hydrogel.

12-14. (canceled)

15. The method of claim 8, wherein said melittin has the amino acid sequence of SEQ ID NO: 1.

16. The method of claim 8, wherein the assay of wound healing activity is selected from the group consisting of shedding of scars, complete re-epithelialization of the wound bed, hydroxyproline content of skin tissue from the wound area, collagen formation and expression levels of collagen 1A1 and 4A1.

17. A method of treating an acute wound to accelerate wound closure and healing, comprising the step of
topically applying to a surface of the wound at least once daily for up to ten days a therapeutically effective amount of a pharmaceutical composition comprising
a carrier selected from the group consisting of a hydrogel, a lotion, a substrate, a wipe and a bandage; and
a plurality of nanoparticles having complexes of melittin, wherein said melittin has the amino acid sequence identity of SEQ ID NO: 1, with one or more non-steroidal anti-inflammatory drug (NSAIDS), distributed therein or thereon, wherein said complexes are ion-pair complexes and wherein a molar ratio of the melittin to NSAIDS in the nanoparticles ranges from 0.1:1.0 to 1.0:0.1, wherein at least one of the one or more NSAIDS is diclofenac;
wherein therapeutic efficacy to accelerate wound closure and healing is determined by measuring wound contraction percentage and/or wound closure.

18. A method of accelerating the process of closure and healing of an acute wound, comprising the step of
topically applying to a surface of the acute wound at least once daily for up to ten days a therapeutically effective amount of a pharmaceutical composition comprising a hydrogel and a plurality of nanoparticles having complexes of melittin, wherein said melittin has the amino acid sequence identity of SEQ ID NO: 1, with diclofenac distributed therein or thereon, wherein said complexes are ion-pair complexes and wherein a molar ratio of the melittin to diclofenac in the nanoparticles is 0.5:1,
wherein therapeutic efficacy to accelerate the process of closure and healing the acute wound is determined by wound closure and/or wound contraction percentage as a measure of the wound closure, and optionally by at least one measure selected from the group consisting of shedding of scars, complete re-epithelialization of the wound bed, hydroxyproline content of skin tissue from the wound area, collagen formation and expression levels of collagen 1A1 and 4A1.

19. The method of claim 8 further comprising performing an assay of wound healing activity one or more times during the topically applying step.


SEQUENCE LISTING (7 sequences)

SEQ ID NO: 1 - protein, 26 aa, Apis mellifera
GIGAVLKVLTTGLPALISWIKRKRQQ

SEQ ID NO: 2 - DNA, 22 bp, Artificial Sequence
  Synthetic primer
atcagcccaaaccccaaggaga

SEQ ID NO: 3 - DNA, 22 bp, Artificial Sequence
  Synthetic primer
cgcaggaaggtcagctggatag

SEQ ID NO: 4 - DNA, 19 bp, Artificial Sequence
  Synthetic primer
cgctgcgaagggtgattgt

SEQ ID NO: 5 - DNA, 21 bp, Artificial Sequence
  Synthetic primer
aaaagggtgatgctggagaac

SEQ ID NO: 6 - DNA, 22 bp, Artificial Sequence
  Synthetic primer
ccattcttccacctttgatgct

SEQ ID NO: 7 - DNA, 24 bp, Artificial Sequence
  Synthetic primer
tgttgctgtagccatattcattgt